(19) United States
(12) Patent Application Publication
(10) Pub. No.: US 2026/0021047 A1
(43) Pub. Date: Jan. 22, 2026

(54) ORAL FORMULATIONS OF DEURUXOLITINIB

(71) Applicant: Sun Pharmaceutical Industries, Inc., Princeton, NJ (US)

(72) Inventors: Brett D. GROTBECK, Lexington, MA (US); Jenisha GANDHI, Lexington, MA (US)

(21) Appl. No.: 19/273,752

(22) Filed: Jul. 18, 2025

Related U.S. Application Data
(60) Provisional application No. 63/673,603, filed on Jul. 19, 2024.
(60) Provisional application No. 63/675,645, filed on Jul. 25, 2024.
(60) Provisional application No. 63/749,111, filed on Jan. 24, 2025.

(51) Int. Cl. A61K 9/20; A61K 9/28; A61K 31/519

(52) U.S. Cl. CPC A61K 9/2077; A61K 9/2009; A61K 9/2018; A61K 9/2027; A61K 9/2054; A61K 9/284; A61K 31/519

(57) ABSTRACT

The present disclosure provides oral dosage forms comprising about 8.75% w/w deuruxolitinib phosphate, about 50% to about 60% w/w microcrystalline cellulose, about 25% to about 35% w/w lactose monohydrate, about 3% to about 6% w/w polyvinylpyrrolidone, about 2% to about 4% w/w hydroxypropyl cellulose, about 0.25% to about 0.75% w/w colloidal silicon dioxide, and about 0.25% to about 0.75% w/w magnesium stearate.

### FIELD OF THE INVENTION

The present disclosure is directed to oral dosage forms comprising about 8.75% w/w deuruxolitinib phosphate, about 50% to about 60% w/w microcrystalline cellulose, about 25% to about 35% w/w lactose monohydrate, about 3% to about 6% w/w polyvinylpyrrolidone, about 2% to about 4% w/w hydroxypropyl cellulose, about 0.25% to about 0.75% w/w colloidal silicon dioxide, and about 0.25% to about 0.75% w/w magnesium stearate, exclusive of film coating.

### BACKGROUND OF THE INVENTION

Deuruxolitinib phosphate, known by the chemical name 1H-pyrazole-1-propanenitrile, β-(cyclopentyl-2,2,3,3,4,4,5,5-d₈)-4-(7H-pyrrolo[2,3-d]pyrimidin-4-yl)-, (βR)-, phosphate (1:1), is a Janus kinase (JAK) inhibitor. U.S. Pat. No. 10,561,659 reports the use of deuruxolitinib phosphate for the treatment of hair loss disorders. PCT publication Nos. WO2020163653 and WO2022/036030 reports processes for the preparation of deuruxolitinib.

For administration of deuruxolitinib and other actives, the oral route of administration is the most preferred route. The advantages of developing oral dosage forms of medication include non-invasiveness, increased patient compliance, and convenience of drug administration. Dosage forms suitable for oral administration may take the form of tablets. However, tablets and other oral forms of active drugs do have potential disadvantages that must be taken into consideration when developing a suitable formulation. Oral dosage forms, such as tablets, must maintain the active pharmaceutical ingredient stability and support bioavailability. However, tablets are more likely to break down inconsistently, which can decrease the drug's effectiveness and overall absorption. Further, tablets with poor friability and hardness are prone to chipping, cracking, sticking, and capping during manufacture, transportation, and storage. As such, it is desirable to produce tablets and other oral dosage forms that are resistant to breakage and fracture while still capable of dissolving in the gastrointestinal tract of the subject administered the oral dosage forms.

### SUMMARY OF THE INVENTION

The disclosure is directed to oral dosage form of 1H-pyrazole-1-propanenitrile, j3-(cyclopentyl-2,2,3,3,4,4,5,5-d8)-4-(7H-pyrrolo[2,3-d]pyrimidin-4-yl)-, (βR)-, phosphate (1:1), which is referred to herein as “deuruxolitinib phosphate,” wherein the oral dosage form comprises deuruxolitinib phosphate and pharmaceutically acceptable carrier material, wherein the dosage form comprises less than 0.05% wt/wt CTP-543-ZH.

In some embodiments, the oral dosage form the pharmaceutically acceptable carrier material comprises of microcrystalline cellulose, lactose monohydrate, polyvinylpyrrolidone, hydroxypropyl cellulose, colloidal silicon dioxide and magnesium stearate.

In some embodiments, the oral dosage form comprises of pharmaceutically acceptable carrier material comprising a low peroxide grade polyvinylpyrrolidone.

In some embodiments, the oral dosage form comprises about 8.75% w/w deuruxolitinib phosphate, about 50% to about 60% w/w microcrystalline cellulose, about 25% to about 35% w/w lactose monohydrate, about 3% to about 6% w/w polyvinylpyrrolidone, about 2% to about 4% w/w hydroxypropyl cellulose, about 0.25% to about 0.75% w/w colloidal silicon dioxide, and about 0.25% to about 0.75% w/w magnesium stearate.

In some embodiments, the oral dosage form of the disclosure comprises about 8.75% w/w deuruxolitinib phosphate, about 52% w/w microcrystalline cellulose, about 30% w/w lactose monohydrate, about 5% w/w polyvinylpyrrolidone, about 3% w/w hydroxypropyl cellulose, about 0.5% w/w colloidal silicon dioxide, and about 0.5% w/w magnesium stearate.

In some embodiments, the polyvinylpyrrolidone is a low peroxide grade polyvinylpyrrolidone.

In some embodiments, the oral dosage form includes:
a. about 8.75% w/w deuruxolitinib phosphate;
b. about 50% to about 60% w/w microcrystalline cellulose;
c. about 25% to about 35% w/w lactose monohydrate;
d. about 3% to about 6% w/w polyvinylpyrrolidone;
e. about 2% to about 4% w/w hydroxypropyl cellulose;
f. about 0.25% to about 0.75% w/w colloidal silicon dioxide; and
g. about 0.25% to about 0.75% w/w magnesium stearate.

In some embodiments, the oral dosage form includes:
i. about 8.75% w/w deuruxolitinib phosphate;
ii. about 52% w/w microcrystalline cellulose;
iii. about 30% w/w lactose monohydrate;
iv. about 5% w/w polyvinylpyrrolidone;
v. about 3% w/w hydroxypropyl cellulose;
vi. about 0.5% colloidal silicon dioxide; and
vii. about 0.5% magnesium stearate.

In some embodiments, the pharmaceutically acceptable carrier in the oral dosage form comprises a low peroxide grade polyvinylpyrrolidone.

In some embodiments, the oral dosage form has a total weight of 100 mg to 1000 mg. In some embodiments, the microcrystalline cellulose in the oral dosage form is in intragranular form and extragranular form. In some embodiments, the oral dosage form comprises about 30% w/w to about 40% w/w intragranular microcrystalline cellulose and about 18% w/w to about 25% w/w extragranular microcrystalline cellulose (e.g., about 19% extragranular microcrystalline cellulose). In some embodiments, the oral dosage form comprises about 33-34% (e.g., about 33%) w/w intragranular microcrystalline cellulose and about 19% w/w extragranular microcrystalline cellulose.

In some embodiments, the oral dosage form does not comprise sodium starch glycolate.

In some embodiments, the polyvinylpyrrolidone in the oral dosage form is a low peroxide grade polyvinylpyrrolidone.

In some embodiments, the oral dosage form is in a tablet. In some embodiments, the oral dosage form is suitable for twice a day administration.

In some embodiments, the oral dosage form comprises less than 0.05% wt/wt CTP-543-ZH after storage for 1 week at 25° C. at 50% relative humidity. In some of the embodiment the oral dosage form comprises less than 0.05% wt/wt CTP-543-ZH after storage for 1 week at 40° C. at 75% relative humidity. In some of the embodiment the oral dosage form comprises less than 0.05% wt/wt CTP-543-ZH after storage for 1 month at 25° C. at 50% relative humidity. In some of the embodiment the oral dosage form comprises less than 0.05% wt/wt CTP-543-ZH after storage for 1 month at 40° C. at 75% relative humidity. In some of the embodiment the oral dosage form comprises less than 0.05% wt/wt CTP-543-ZH after storage for 6 months at 25° C. at 50% relative humidity. In some of the embodiment the oral dosage form comprises less than 0.05% wt/wt CTP-543-ZH after storage for 6 months at 40° C. at 75% relative humidity.

In some of the embodiment, the oral dosage form comprises less than 0.01% wt/wt CTP-543-ZH.

In some of the embodiment, the oral dosage form comprises less than 0.01% wt/wt CTP-543-ZH after storage for 1 week at 25° C. at 50% relative humidity. In some of the embodiment the oral dosage form of comprises less than 0.01% wt/wt CTP-543-ZH after storage for 1 week at 40° C. at 7 5% relative humidity. In some of the embodiment the oral dosage form comprises less than 0.01% wt/wt CTP-543-ZH after storage for 1 month at 25° C. at 50% relative humidity. In some of the embodiment the oral dosage form comprises less than 0.01% wt/wt CTP-543-ZH after storage for 1 month at 40° C. at 75% relative humidity. In some of the embodiment the oral dosage form comprises less than 0.01% wt/wt CTP-543-ZH after storage. In some of the embodiment the oral dosage form comprises less than 0.01% wt/wt CTP-543-ZH after storage for 6 months at 25° C. at 50% relative humidity.

In some of the embodiment the oral dosage form comprises less than 0.01% wt/wt CTP-543-ZH after storage for 6 months at 40° C. at 75% relative humidity.

In some embodiments, the disclosure provides a process for the preparation of the oral dosage forms as described herein that comprises (a) combining deuruxolitinib phosphate, microcrystalline cellulose, lactose monohydrate, hydroxypropyl cellulose, and polyvinylpyrrolidone; (b) wet granulating the combination of (a) to form particles; (c) blending the particles formed with microcrystalline cellulose, colloidal silicon dioxide and magnesium stearate to form a blend; and (d) compressing the blend into a tablet.

In some embodiments, the disclosure provides a coated tablet comprising a tablet core and an outer coating layer, wherein the tablet core comprises about 8.75% w/w deuruxolitinib phosphate, about 52% w/w microcrystalline cellulose, about 30% w/w lactose monohydrate, about 5% w/w polyvinylpyrrolidone, about 3% w/w hydroxypropyl cellulose, about 0.5% colloidal silicon dioxide, and about 0.5% magnesium stearate.

In some embodiments, the polyvinylpyrrolidone in the coated tablet is a low peroxide grade polyvinylpyrrolidone. In some embodiments, the outer coating layer comprises Opadry® amb II. In some embodiments, the coated tablet has a total weight of 100 mg to 1000 mg.

In some embodiments, the disclosure provides a method of treating a hair disorder in a human subject, comprising administering to the human subject the oral dosage form or the coated table as described herein. In some embodiments, the hair loss disorder is alopecia areata. In some embodiments, the oral dosage form or the coated tablet is administered twice a day.

### DETAILED DESCRIPTION OF THE INVENTION

The present disclosure relates to a pharmaceutical formulation for oral administration comprising deuruxolitinib phosphate, methods of making pharmaceutical compositions comprising deuruxolitinib phosphate, and the use of deuruxolitinib phosphate for treating, preventing, or ameliorating a disease or condition comprising administering an oral dosage form of the disclosure.

The term 1H-pyrazole-1-propanenitrile, β-(cyclopentyl-2,2,3,3,4,4,5,5-d₈)-4-(7H-pyrrolo[2,3-d]pyrimidin-4-yl)-, (βR)-, phosphate (1:1) can be represented by a compound of Formula I:

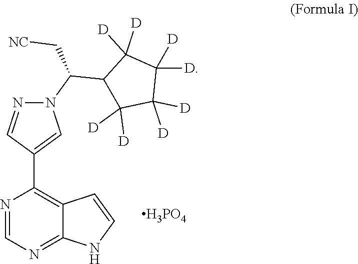

1H-pyrazole-1-propanenitrile, β-(cyclopentyl-2,2,3,3,4,4,5,5-d₈)-4-(7H-pyrrolo[2,3-d]pyrimidin-4-yl)-, (βR)-, phosphate (1:1) is also interchangeably referred to herein as deuruxolitinib phosphate and deuruxolitinib phosphate (1:1). One of skill in the art will understand that for each of the positions shown in Formula I as deuterium (i.e., “D”), deuterium may not be incorporated in 100% of the positions and would still fall within the scope of the term deuruxolitinib phosphate. In some embodiments, the term “deuruxolitinib phosphate” refers to a material in which given a population of deuruxolitinib phosphate molecules, e.g., a preparation of deuruxolitinib phosphate, at least 90% of each specified deuterated position comprises deuterium, or at least 91%, at least 92%, at least 93%, at least 94%, at least 95%, at least 96%, at least 97%, at least 98%, or at least 99% of each specified deuterated position comprises deuterium as determined by ¹H-NMR.

In some embodiments, the disclosure is directed to pharmaceutical compositions comprising deuruxolitinib phosphate, such a pharmaceutical composition formulated for oral administration to a subject for treating, preventing, or ameliorating a disease or condition. In particular, in some embodiments, oral dosage forms of deuruxolitinib phosphate and the combination of carrier materials provides tablets that can exhibit, among other properties, improved chemical stability, hardness, crushing strength, porosity, and/or friability properties.

The present disclosure provides new oral dosage forms of deuruxolitinib phosphate. In some embodiments, oral dosage forms are capsules, tablets, wafers, or chewable tablets. In certain embodiments, the oral dosage forms are tablets or coated tablets. The present application describes the chemical and physical characteristics of these oral dosage forms, and discloses methods for making these oral dosage forms.

In some embodiment, the present disclosure provides an oral dosage form comprising deuruxolitinib phosphate and pharmaceutically acceptable carrier material, wherein the oral dosage form comprises less than 0.05% wt/wt CTP-543-ZH.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps. It is contemplated that any embodiment discussed in this specification can be implemented with respect to polymorph, methods, and/or kits of the present disclosure. Furthermore, the polymorph and/or kits of the present disclosure can be used to achieve the methods of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

As used herein, the term “carrier materials” refers to one or more non-active, non-toxic pharmaceutically acceptable carriers, binders, diluents, excipients, or adjuvants. The carrier materials are acceptable in the sense of being compatible with the other ingredients of the composition and are not deleterious to the recipient. The pharmaceutical compositions of the present invention can be adapted for administration by any suitable route by selection of appropriate carrier materials and a dosage of a compound of the disclosure effective for the treatment intended. For example, these compositions can be prepared in a form suitable for administration orally. Accordingly, the carrier material employed can be a solid, and may be formulated with the compound as a unit-dose composition, for example, a tablet. Such pharmaceutical compositions of the disclosure can be prepared by any of the well-known techniques of pharmacy, consisting essentially of admixing the components.

The term “pharmaceutically acceptable” as used herein, refers to a component that is, within the scope of sound medical judgement, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to the recipient, is capable of providing, either directly or indirectly, a compound of this disclosure.

The term “pharmaceutical composition” as used herein refer to a compound or composition capable of inducing a desired therapeutic effect when properly administered to a subject.

As used herein, the term “room temperature” generally refers to 4° C. to 30° C., 18° C. to 22° C., 19° C. to 22° C., 20° C. to 22° C. or 20±5° C.

The term “treating” or “treatment” refers to administering a compound or pharmaceutical composition to a subject in order to affect an alteration or improvement of a disease, disorder, or condition in the subject. The terms “treatment” or “treat” as used herein may refer to both therapeutic treatment and prophylactic or preventative measures. Those in need of treatment include subjects having a disease or condition as well as those prone to having disease or condition or those for which a disease or condition is to be prevented.

The terms “subject” is used herein to refer to a mammalian subject. In some embodiments, the “subject” is a human, domestic animals, farm animals, sports animals, and zoo animals, e.g., humans, non-human primates, dogs, cats, guinea pigs, rabbits, rats, mice, horses, cattle, etc. In some embodiments, the subject is a human. In certain embodiments, the subject is a human. In certain embodiments, the human subject is an adult (i.e., at least 18 years old). In certain embodiments, the human subject is an adolescent (i.e., from 12 years to less than 18 years old). In certain embodiments, the human subject is a pediatric subject (i.e., from 6 years to less than 12 years old).

The disclosure herein provides a pharmaceutical composition containing a pharmaceutically acceptable salt form of deuruxolitinib. In some embodiments, the pharmaceutically acceptable salt form deuruxolitinib is the phosphate salt form, such as the deuruxolitinib phosphate form described above. The selection and combination of carrier materials used in the pharmaceutical compositions of the present invention provides compositions exhibiting improved performance with respect to, among other properties, chemical stability, hardness, porosity, and friability properties. In some embodiments, the pharmaceutical composition is an oral dosage form containing deuruxolitinib phosphate. In some embodiments, the oral dosage form contains about 8.7% to about 9% weight by weight (w/w) deuruxolitinib phosphate, e.g., 8.7%, 8.75%, 8.8%, 8.85%, 8.9%, or 9% deuruxolitinib phosphate. In some embodiments, the oral dosage form contains about 8.75% weight by weight (w/w) deuruxolitinib phosphate or about 8.85% w/w deuruxolitinib phosphate.

Pharmaceutically acceptable carrier materials (e.g., carriers, lubricants, glidants, binders, diluents, excipients, or adjuvants) that may be used in the pharmaceutical compositions of this disclosure include, but are not limited to, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In some embodiments, the pharmaceutical composition includes the deuruxolitinib phosphate and one or more pharmaceutically acceptable carrier materials, such as binders, diluents, glidants, and lubricants. In some embodiments, the one or more pharmaceutically acceptable carrier materials include, but are not limited to colloidal silicon dioxide, corn starch, hydroxypropyl cellulose, lactose, lactose monohydrate, magnesium stearate, microcrystalline cellulose, and polyvinylpyrrolidone (povidone).

In some embodiments, dosage forms, such as oral dosage forms are provided, which include deuruxolitinib phosphate together with one or more of microcrystalline cellulose, lactose monohydrate, polyvinylpyrrolidone, hydroxypropyl cellulose, colloidal silicon dioxide, and/or magnesium stearate. In some embodiments, the oral dosage form contains about 50% to about 60% w/w microcrystalline cellulose or 52% to 55% w/w microcrystalline cellulose. In some embodiments, the oral dosage form contains about 25% to about 35% (e.g., 30%) w/w lactose monohydrate or 30% to 35% w/w lactose monohydrate. In some embodiments, the oral dosage form contains about 3% to about 6%, or about 5% to about 7% w/w polyvinylpyrrolidone or 5% to 7% w/w polyvinylpyrrolidone. In some embodiments, the oral dosage form contains about 2% to about 4%, or about 3% to about 4% w/w hydroxypropyl cellulose or about 3% to about 4% w/w hydroxypropyl cellulose. In some embodiments, the oral dosage form contains about 0.25% to about 0.75% w/w colloidal silicon dioxide or 0.4% to 0.6% colloidal silicon dioxide. In some embodiments, the oral dosage form contains about 0.25% to about 0.75% w/w magnesium stearate or 0.4% to 0.6 w/w magnesium stearate.

In some embodiments, the oral dosage form contains about 8.75% w/w deuruxolitinib phosphate, about 50% to about 60% w/w microcrystalline cellulose, about 25% to about 35% w/w lactose monohydrate, about 3% to about 6% or about 5% to about 7% w/w polyvinylpyrrolidone, about 2% to about 4%, or about 3% to about 4% w/w hydroxypropyl cellulose, about 0.25% to about 0.75% w/w colloidal silicon dioxide, and about 0.25% to about 0.75% w/w magnesium stearate. In some embodiments, the oral dosage form contains about 8.75% w/w deuruxolitinib phosphate together with about 50% to about 60% w/w microcrystalline cellulose, e.g., 50%, 51%, 52%, 53%, 54%, 55%, 56%, 57%, 58%, 59%, or 60% w/w microcrystalline cellulose; about 30% to about 40% w/w lactose monohydrate, e.g., 30%, 31%, 32%, 33%, 34%, 35%, 36%, 37%, 38%, 39%, or 40% w/w lactose monohydrate; about 5% to about 7% w/w polyvinylpyrrolidone, e.g., 5%, 6%, or 7% w/w polyvinylpyrrolidone; about 3% to about 4% w/w hydroxypropyl cellulose, e.g., 3.0%, 3.1%, 3.2%, 3.3%, 3.4%, 3.5%, 3.6%, 3.7%, 3.8%, 3.9%, or 4% w/w hydroxypropyl cellulose; about 0.25% to about 0.75% w/w colloidal silicon dioxide, e.g., 0.25%, 0.3%, 0.35%, 0.4%, 0.45%, 0.5%, 0.55%, 0.6%, 0.65%, 0.7%, or 0.75% w/w colloidal silicon dioxide; and about 0.25% to about 0.75% w/w magnesium stearate, e.g., 0.25%, 0.3%, 0.35%, 0.4%, 0.45%, 0.5%, 0.55%, 0.6%, 0.65%, 0.7%, or 0.75% w/w magnesium stearate. In some embodiments, the oral dosage form may have a total weight of about 100 mg to about 1000 mg (for example, about 100 mg to about 130 mg). In some embodiment, the oral dosage form may have a total weight of about 100 mg to about 200 mg, about 200 mg to about 300 mg, about 300 mg to 400 mg, about 400 mg to about 500 mg, about 500 mg to about 600 mg, about 600 mg to about 700 mg, about 700 mg to about 800 mg, about 800 mg to about 900 mg, or about 900 mg to about 1000 mg. In some embodiments, the oral dosage form may have a total weight of 102.5 mg, 105 mg, 105.5 mg, 110 mg, 112.5 mg, 115 mg, 115.5 mg, 120 mg, 122.5 mg, 125 mg, or 125.5 mg, or 130 mg. In some embodiments, the oral dosage form has a total weight of about 120 mg.

In some embodiments, the oral dosage form contains about 4% to about 50% deuruxolitinib phosphate, or about 4% to about 45% deuruxolitinib phosphate, or about 4% to about 40% deuruxolitinib phosphate, or about 4% to about 35% deuruxolitinib phosphate, or about 4% to about 30% deuruxolitinib phosphate, or about 4% to about 25% deuruxolitinib phosphate, or about 4% to about 20% deuruxolitinib phosphate, or about 4% to about 15% deuruxolitinib phosphate, or about 4% to about 10% deuruxolitinib phosphate, or about 5% to about 50% deuruxolitinib phosphate, or about 10% to about 50% deuruxolitinib phosphate, or about 10% to about 40% deuruxolitinib phosphate, or about 10% to about 30% deuruxolitinib phosphate, or about 10% to about 20% deuruxolitinib phosphate, or about 20% to about 50% deuruxolitinib phosphate, or about 20% to about 40% deuruxolitinib phosphate, or about 20% to about 30% deuruxolitinib phosphate, or about 30% to about 50% deuruxolitinib phosphate, or about 30% to about 40% deuruxolitinib phosphate, or about 40% to about 50% deuruxolitinib phosphate, or about 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% deuruxolitinib phosphate.

In some embodiments, the oral dosage form contains about 4% to about 20% deuruxolitinib phosphate, about 50% to about 60% microcrystalline cellulose, about 25% to about 35% lactose monohydrate, about 3% to about 6% polyvinylpyrrolidone, about 0.25% to about 0.75% colloidal silicon dioxide, and about 0.25% to about 0.75% hydroxypropyl cellulose.

In some embodiments, the oral dosage form contains about 8.75% w/w deuruxolitinib phosphate (e.g., 8.75% w/w deuruxolitinib phosphate), about 52% w/w microcrystalline cellulose (e.g., 52% w/w microcrystalline cellulose), about 30% w/w lactose monohydrate (e.g., 30% w/w lactose monohydrate), about 5% w/w polyvinylpyrrolidone (e.g., 5% w/w polyvinylpyrrolidone), about 3% w/w hydroxypropyl cellulose (3% w/w hydroxypropyl cellulose), about 0.5% w/w colloidal silicon dioxide (e.g., 0.5% w/w colloidal silicon dioxide); and about 0.5% w/w magnesium stearate (e.g., 0.5% w/w magnesium stearate). In such embodiments, the oral dosage form may have a total weight of about 100 mg to about 1000 mg (for example, about 100 mg to about 130 mg). In some embodiments, the oral dosage form may have a total weight of about 100 mg to about 200 mg, about 200 mg to about 300 mg, about 300 mg to 400 mg, about 400 mg to about 500 mg, about 500 mg to about 600 mg, about 600 mg to about 700 mg, about 700 mg to about 800 mg, about 800 mg to about 900 mg, or about 900 mg to about 1000 mg. In some embodiments, the oral dosage form may have a total weight of 100 mg, 102.5 mg, 105 mg, 105.5 mg, 110 mg, 112.5 mg, 115 mg, 115.5 mg, 120 mg, 122.5 mg, 125 mg, 125.5 mg, or 130 mg.

In some embodiments, the oral dosage forms of the disclosure include microcrystalline cellulose, which may function as binding agent and as a disintegrating agent. A disintegrating agent enables a tablet formulation to break apart when placed in an aqueous environment. As discussed elsewhere herein, some oral dosage forms of the disclosure may be produced by a process that includes wet granulation (e.g., tablet forms). In some embodiments, microcrystalline cellulose may be added before the granulation process, and the microcrystalline cellulose is sometimes referred to herein as “intragranular.” In some embodiments, microcrystalline cellulose may be added after the granulation process and prior to compression of the oral dosage form, and the microcrystalline cellulose is sometimes referred to herein as “extragranular.” In some embodiments, microcrystalline cellulose is added in the granulation process and after the granulation process, but prior to compression of the oral dosage form. In some embodiments, the oral dosage form contains intragranular microcrystalline cellulose. In some embodiments, the oral dosage form contains extragranular microcrystalline cellulose. In some embodiments, both intragranular microcrystalline cellulose and extragranular microcrystalline cellulose are present in the oral dosage form.

In some embodiments, the oral dosage form contains about 30% w/w to about 40% w/w intragranular microcrystalline cellulose and about 18% w/w to about 25% w/w extragranular microcrystalline cellulose (e.g., about 19% extragranular microcrystalline cellulose). In some embodiments, the oral dosage form contains about 30% w/w to about 40% w/w intragranular microcrystalline cellulose, e.g., 30%, 31%, 32%, 33%, 34%, 35%, 36%, 37%, 38%, 39%, or 40% w/w intragranular microcrystalline cellulose; and about 18% w/w to about 30% w/w extragranular microcrystalline cellulose, e.g., 18%, 19%, 20%, 21%, 22%, 23%, 24%, 25%, 26%, 27%, 28%, 29%, or 30% w/w extragranular microcrystalline cellulose. In some embodiments, the oral dosage form contains 32% to 38% or 33%-34% (e.g., about 33%) w/w intragranular microcrystalline cellulose; and 18% to 28% or 18% to 20% w/w extragranular microcrystalline cellulose. In some embodiments, the oral dosage form contains about 33%-34% (e.g., about 33%) w/w intragranular microcrystalline cellulose. In some embodiments, the oral dosage form contains about 19% w/w extragranular microcrystalline cellulose. In some embodiments, the oral dosage form contains about 33% w/w intragranular microcrystalline cellulose and about 19% w/w extragranular microcrystalline cellulose.

In some embodiments, oral dosage forms do not include one or more of ferric oxide, crospovidone, or glyceryl dibehenate.

The oral dosage forms provided herein will also exhibit physical and chemical properties, including, but not limited to, hardness, disintegration, tapped density, porosity, friability, stability, solubility, and the like. In some embodiments, the combination of the components in the oral dosage forms, such as the oral dosage forms described above, impart to the oral dosage form improvements in one or more of these physical and chemical properties as compared to oral dosage forms in the art.

In some embodiments, the oral dosage forms of the present disclosure exhibit improved porosity. While not intending to be bound by theory, porosity impacts how easily a solvent, such as water, can penetrate the solid matrix of an oral dosage form, such as a tablet. Porosity of the oral dosage forms can be measured by art-standard techniques (i.e., in a density analyzer). In some embodiments, the porosity of the oral dosage forms 15-25%, or about 18-20%.

In some embodiments, the oral dosage forms of the present disclosure exhibit improved hardness. In some embodiments, the hardness of the oral dosage forms is about 4.5-7.5 kp, e.g., about 5.5 kp.

Another physical characteristic of oral dosage forms is friability, which is a tendency of an oral dosage form, such as a tablet or capsule, to chip, crumble, or break when subjected to stress during, for example, transportation and handling. As will be readily understood by one having ordinary skill in the art, tablets and other sold oral dosage forms need to be hard enough to withstand compression and other pressures exerted during manufacture and storage so that they do not break up in the bottle or other storage container while, at the same time, exhibiting sufficient disintegration so that they dissolve in the gastrointestinal tract. Art-standard testing can be performed to measure friability of an oral dosage form. For instance, a non-limiting embodiment of a basic measuring apparatus may include a friability drum and motor capable of rotating the friability drum at about 25 revolutions per minute (“rpm”), or more, where the friability drum has an internal diameter of 250 mm and 300 mm and a depth of about 30 mm and 40 mm. In some embodiments, the friability tester may include an abrasion drum. In some embodiments, the friability of uncoated oral dosage forms of the disclosure are measured. In some embodiments, the friability of coated oral dosage forms of the disclosure are measured.

To measure the friability of an oral dosage form, one or more (e.g., a plurality of tablets) units of an oral dosage form are placed inside the interior of the friability drum and will rotate and collide with the walls of the drum or with other oral dosage forms present in the apparatus. The friability is determined by the percentage of weight loss exhibited by the oral dosage form (i.e., the amount of solid that has broken off or has been crushed due to the testing apparatus). In some embodiments, an average weight loss of less than 1% is considered acceptable (for example, the average weight loss for 6.5 g of tablets).

In some embodiments, the oral dosage forms of the present disclosure exhibit improved friability. In some embodiments, the friability of the oral dosage forms exhibit a weight loss of less than 1% when subjected to friability testing at about 24 rpm to about 26 rpm. In some embodiments, the friability of the oral dosage forms of the disclosure exhibit a weight loss of less than about 0.5% when subjected to friability testing at about 24 rpm to about 26 rpm. In some embodiments, the friability of the oral dosage forms of the disclosure exhibit a weight loss of less than about 0.2% when subjected to friability testing at about 24 rpm to about 26 rpm.

In some embodiments, the oral dosage forms of the present disclosure exhibit increased solubility in water. In some embodiments, the oral dosage forms of the present disclosure having a total weight of 100 mg to 1000 mg (e.g., 100 mg to 130 mg) dissolve in water. In such embodiments, at least about 80% of the 100 mg to 1000 mg (e.g., 100 mg to 130 mg) oral dosage forms dissolve in water in less than 30 minutes

In some embodiments, the oral dosage form is manufactured as a dissolving tablet. As one having ordinary skill in the art would appreciate, dissolving tablets readily dissolve in water.

In certain embodiments, the pharmaceutical compositions of the disclosure are administered orally as oral dosage forms. Pharmaceutical compositions of the present disclosure suitable for oral administration may be presented as discrete units such as, but not limited to, capsules, sachets, wafers, or tablets, each containing the ingredients described above. In some embodiments, the oral dosage form is a tablet. In some embodiments, the oral dosage form is a coated tablet.

In some embodiments, an oral dosage form of the present disclosure is provided in the form of a tablet. In some embodiments provided herein is a coated tablet that includes a tablet core and an outer coating layer. In such embodiments, the tablet core may be made from deuruxolitinib phosphate together with one or more of microcrystalline cellulose, lactose monohydrate, polyvinylpyrrolidone, hydroxypropyl cellulose, colloidal silicon dioxide, and magnesium stearate. In some embodiments, the tablet core comprises about 8.75% w/w deuruxolitinib phosphate, about 50% to about 60% w/w microcrystalline cellulose, about 25% to about 35% w/w lactose monohydrate, about 3% to about 6% w/w polyvinylpyrrolidone, about 2% to about 4% w/w hydroxypropyl cellulose, about 0.25% to about 0.75% w/w colloidal silicon dioxide, and about 0.25% to about 0.75% w/w magnesium stearate. In some embodiments, the tablet core comprises about 8.75% w/w deuruxolitinib phosphate (e.g., 8.75% w/w deuruxolitinib phosphate), about 52% w/w microcrystalline cellulose (e.g., 52.25% w/w microcrystalline cellulose), about 30% w/w lactose monohydrate (e.g., 30% w/w lactose monohydrate), about 5% w/w polyvinylpyrrolidone (e.g., 5% w/w polyvinylpyrrolidone), about 3% w/w hydroxypropyl cellulose (3% w/w hydroxypropyl cellulose), about 0.5% w/w colloidal silicon dioxide (e.g., 0.5% w/w colloidal silicon dioxide), and about 0.5% w/w magnesium stearate (e.g., 0.5% w/w magnesium stearate). In such embodiments, the outer coating layer may comprise a high performance moisture barrier film coating. In some embodiments, the outer coating layer comprises Opadry® amb II coating. In some embodiments, the coated tablet may have a total weight of about 100 mg to about 1000 mg. In some embodiments, the coated tablet may have a total weight of about 100 mg to about 200 mg, about 200 mg to about 300 mg, about 300 mg to 400 mg, about 400 mg to about 500 mg, about 500 mg to about 600 mg, about 600 mg to about 700 mg, about 700 mg to about 800 mg, about 800 mg to about 900 mg, or about 900 mg to about 1000 mg. In some embodiments, the coated tablet may have a total weight of 100 mg, 102.5 mg, 105 mg, 105.5 mg, 110 mg, 112.5 mg, 115 mg, 115.5 mg, 120 mg, 122.5 mg, 125 mg, or 125.5 mg, or 130 mg.

In some embodiments, the dosage forms disclosed herein comprise a pharmaceutically acceptable carrier. In some embodiments, the pharmaceutically acceptable carriers use one or more methods of making, and/or are made by one or more manufacturers and may contain low concentrations of an oxidizing agent, e.g., peroxide. Thus, in some embodiments, the pharmaceutical composition of the disclosure comprises a pharmaceutically acceptable carrier and deuruxolitinib, wherein the composition contains a low concentrations of an oxidizing agent, e.g., a low peroxide grade pharmaceutically acceptable carrier, e.g., polyvinylpyrrolidone. In some embodiments, one of the pharmaceutically acceptable carrier comprises low concentrations of an oxidizing agent, e.g., peroxide. In some embodiments, two or more of the pharmaceutically acceptable carrier comprises low concentrations of an oxidizing agent, e.g., peroxide. In some embodiments, each of the pharmaceutically acceptable carriers comprises low concentrations of an oxidizing agent, e.g., peroxide.

In some embodiments, suitable grades of polyvinylpyrrolidone (povidone) for use in the oral dosage forms of the disclosure include, but are not limited to, povidone K25, povidone K30, povidone K30 LP, povidone K90, povidone C, and the like. In some embodiments, the povidone is povidone K30. In some embodiments, the povidone is a low peroxide povidone, such as povidone K30 LP (e.g., Kollidon® 30 LP). The disclosure provides that a low peroxide grade povidone binder can decrease the total amount of degradation products in the oral dosage forms of the disclosure. In some embodiments, oral dosage forms comprising low peroxide grade povidone contain less total degradation products compared to oral dosage forms comprising higher peroxide grade povidone. In some embodiments, oral dosage forms comprising low peroxide grade povidone contain less total degradation products after manufacture/formulation compared to oral dosage forms comprising higher peroxide grade povidone. In some embodiments, oral dosage forms comprising low peroxide grade povidone contain less total degradation products after storage compared to oral dosage forms comprising higher peroxide grade povidone.

In some embodiments, “low peroxide grade” povidone refers to povidone comprising less than about 100 ppm peroxide (e.g., hydrogen peroxide), e.g., between 0 ppm and 100 ppm, or between about 10 ppm and about 100 ppm, or between about 20 ppm and about 50 ppm, or between about 10 ppm and about 20 ppm, or between 0 ppm and about 25 ppm. It being understood that the peroxide levels will increase in the povidone over time depending on handling and storage conditions. In some embodiments, “low peroxide grade” povidone refers to povidone comprising less than about 100 ppm peroxide, or less than about 50 ppm, or less than about 25 ppm, or less than about 10 ppm. In some embodiments, low peroxide grade povidone contains less than about 25 ppm peroxide.

In some embodiments, the povidone binder can decrease the total degradation products in the oral dosage forms of the disclosure, which degradation products can be measured using art standard means, such as, but not limited to high performance liquid chromatography (HPLC). Degradation products can be formed during the manufacturing/formulation process or during shipping, handling, and/or storage. When using HPLC, the weight percentage of various impurities and/or total impurities can be determined by measuring relative retention times of the various analytes as they are eluted from the column of the HPLC and comparing the values to reference standards for deuruxolitinib phosphate and/or degradation products. In this manner, the weight percentage of an impurity of interest (i.e., a degradation product) can be determined. In addition, the weight percentage of total degradation products can be determined as these analytes will have different retention times compared to deuruxolitinib phosphate.

In some embodiments, the oral dosage forms of the disclosure contain about 0.000001% w/w to about 2% w/w total degradation products after storage at room temperature for at least 2 years (e.g., as measured by HPLC), or 0.000001% w/w to 1.5% w/w total degradation products after storage at room temperature for at least 2 years, or 0.000001% w/w to 1% w/w total degradation products after storage at room temperature for at least 2 years, or 0.000001% w/w to 0.5% w/w total degradation products after storage at room temperature for at least 2 years. In some embodiments, the oral dosage forms of the disclosure contain 0.000001% w/w to 2% w/w total degradation products after storage at about 30° C. for at least 1 year, or 0.000001% w/w to 1.5% w/w total degradation products after storage at 30° C. for at least 1 year, or 0.000001% w/w to 1% w/w total degradation products after storage at 30° C. for at least 1 year, or 0.000001% w/w to 0.5% total degradation products after storage at 30° C. for at least 1 year. In some embodiments, the oral dosage forms of the disclosure contain between 0.000001% w/w and 2% w/w total degradation products after storage at about 40° C. for at least 6 months, or 0.000001% w/w to 1.5% w/w total degradation products after storage at 40° C. for at least 6 months, or 0.000001% w/w to 1% w/w total degradation products after storage at 40° C. for at least 6 months, or 0.000001% w/w to 0.5% total degradation products after storage at 430° C. for at least 6 months. In some embodiments, the oral dosage form contains low peroxide grade povidone containing between 0 ppm and about 50 ppm or between 0 ppm and about 25 ppm (e.g., povidone K30 LP).

In some embodiments, the degradation products of the oral dosage forms comprise one or more degradation products, including, but not limited to CTP-543-ZB and/or CTP-543-ZH. The CTP-543-ZB and CTP-543-ZH degradation products have the following structures:

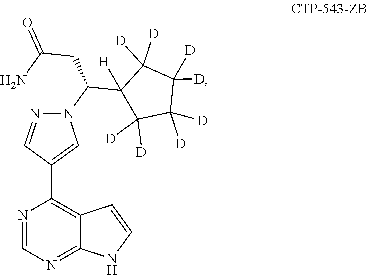

which is also known as (R)-3-(4-(7H-pyrrolo[2,3-d]pyrimidin-4-yl)-1H-pyrazol-1-yl)-3-(cyclopentyl-2,2,3,3,4,4,5,5-d8)propenamide, and

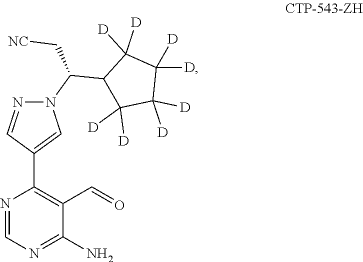

which is also known as (R)-3-(4-(6-amino-5-formylpyrimidin-4-yl)-1H-pyrazol-1-yl)-3-(cyclopentyl-2,2,3,3,4,4,5,5-d8)propanenitrile.

In some embodiments, the oral dosage forms of the disclosure contain 0.000001% w/w to 0.5% w/w CTP-543-ZH, or 0.000001% w/w to 0.1% w/w CTP-543-ZH, or 0.000001% w/w to 0.05% w/w CTP-543-ZH (e.g., as measured by HPLC). In some embodiments, the oral dosage form of the disclosure does not contain a detectable amount of CTP-543-ZH as measured by HPLC using a detection threshold of 0.05% w/w. In some embodiments, the oral dosage form of the disclosure does not contain a detectable amount of CTP-543-ZH as measured by HPLC.

In some embodiments, an oral dosage form is provided herein, the tablet core of the oral dosage form may be made from deuruxolitinib phosphate together with polyvinylpyrrolidone (povidone) and, optionally, one or more of microcrystalline cellulose, colloidal silicon dioxide and magnesium stearate. In some embodiments, the tablet core of the oral dosage for may be made from deuruxolitinib phosphate, polyvinylpyrrolidone (povidone), microcrystalline cellulose, colloidal silicon dioxide and magnesium stearate. In some embodiments, the tablet core comprises about 5% w/w polyvinylpyrrolidone (povidone) (e.g., 5%) and exhibits 0.000001% to 2% w/w total degradation products after storage at room temperature for at least 2 years (e.g., as measured by HPLC), or 0.000001% to 1.5% w/w total degradation products after storage at room temperature for at least 2 years, or 0.000001% to 1% w/w total degradation products after storage at room temperature for at least 2 years, or 0.000001% to 0.5% total degradation products after storage at room temperature for at least 2 years.

In some embodiments, the ratio of CTP-543 to CTP-543-ZH in the oral dosage form is about 10:1 to about 10⁸:1, about 100:1 to about 10⁸:1, about 10³:1 to about 10⁸:1, about 10⁴:1 to about 10⁸:1, or about 10¹:1 to about 10⁸:1 after 1 week at 25° C. at 50% relative humidity. In some embodiments, the ratio of CTP-543 to CTP-543-ZH in the oral dosage form is about 10:1 to about 10⁸:1, about 100:1 to about 10⁸:1, about 10³:1 to about 10⁸:1, about 10⁴:1 to about 10⁸:1, or about 10¹:1 to about 10⁸:1 after 1 month at 25° C. at 50% relative humidity. In some embodiments, the ratio of CTP-543 to CTP-543-ZH in the oral dosage form is about 10:1 to about 10⁸:1, about 100:1 to about 10⁸:1, about 10³:1 to about 10⁸:1, about 10⁴:1 to about 10⁸:1, or about 10¹:1 to about 10⁸:1 after 6 months at 25° C. at 50% relative humidity. In some embodiments, the ratio of CTP-543 to CTP-543-ZH in the oral dosage form is about 10:1 to about 10⁸:1, about 100:1 to about 10⁸:1, about 10³:1 to about 10⁸:1, about 10⁴:1 to about 10⁸:1, or about 10⁵:1 to about 10⁸:1 after 1 year at 25° C. at 50% relative humidity.

In some embodiments, the ratio of CTP-543 to CTP-543-ZH in the oral dosage form is about 10:1 to about 10⁸:1, about 100:1 to about 10⁸:1, about 10³:1 to about 10⁸:1, about 10⁴:1 to about 10⁸:1, or about 10¹:1 to about 10⁸:1 after 1 week at 40° C. at 75% relative humidity. In some embodiments, the ratio of CTP-543 to CTP-543-ZH in the oral dosage form is about 10:1 to about 10⁸:1, about 100:1 to about 10⁸:1, about 10³:1 to about 10⁸:1, about 10⁴:1 to about 10⁸:1, or about 10¹:1 to about 10⁸:1 after 1 month at 40° C. at 75% relative humidity. In some embodiments, the ratio of CTP-543 to CTP-543-ZH in the oral dosage form is about 10:1 to about 10⁸:1, about 100:1 to about 10⁸:1, about 10³:1 to about 10⁸:1, about 10⁴:1 to about 10⁸:1, or about 10¹:1 to about 10⁸:1 after 6 months at 40° C. at 75% relative humidity. In some embodiments, the ratio of CTP-543 to CTP-543-ZH in the oral dosage form is about 10:1 to about 10⁸:1, about 100:1 to about 10⁸:1, about 10³:1 to about 10⁸:1, about 10⁴:1 to about 10⁸:1, or about 10′:1 to about 10⁸:1 after 1 year at 40° C. at 75% relative humidity.

In some embodiments, the amount of CTP-543-ZH is undetectable using standard techniques, but can be detected when using high precision methods, techniques, and instrumentation, or can be detected when the compound of any one of Formulas (I)-(V) is first concentrated before detection.

In some embodiments, the dosage form comprises less than 0.05% wt/wt CTP-543-ZH. In some embodiments, the dosage form comprises less than 0.05% wt/wt CTP-543-ZH after storage for 1 week at 25° C. at 50% relative humidity. In some embodiments, the dosage form comprises less than 0.05% wt/wt CTP-543-ZH after storage for 1 week at 40° C. at 75% relative humidity. In some embodiments, the dosage form comprises less than 0.05% wt/wt CTP-543-ZH after storage for 1 month at 25° C. at 50% relative humidity. In some embodiments, the dosage form comprises less than 0.05% wt/wt CTP-543-ZH after storage for 1 month at 40° C. at 75% relative humidity. In some embodiments, the dosage form comprises less than 0.05% wt/wt CTP-543-ZH after storage for 6 months at 25° C. at 50% relative humidity. In some embodiments, the dosage form comprises less than 0.05% wt/wt CTP-543-ZH after storage for 6 months at 40° C. at 75% relative humidity.

In some embodiments, the dosage form comprises less than 0.01% wt/wt CTP-543-ZH. In some embodiments, the dosage form comprises less than 0.01% wt/wt CTP-543-ZH after storage for 1 week at 25° C. at 50% relative humidity. In some embodiments, the dosage form comprises less than 0.01% wt/wt CTP-543-ZH after storage for 1 week at 40° C. at 75% relative humidity. In some embodiments, the dosage form comprises less than 0.01% wt/wt CTP-543-ZH after storage for 1 month at 25° C. at 50% relative humidity. In some embodiments, the dosage form comprises less than 0.01% wt/wt CTP-543-ZH after storage for 1 month at 40° C. at 75% relative humidity. In some embodiments, the dosage form comprises less than 0.01% wt/wt CTP-543-ZH after storage for 6 months at 25° C. at 50% relative humidity. In some embodiments, the dosage form comprises less than 0.01% wt/wt CTP-543-ZH after storage for 6 months at 40° C. at 75% relative humidity.

In some embodiments, the tablet core comprises about 5% w/w povidone containing between 0 and about 25 ppm peroxides (e.g., 5%) and exhibits 0.000001% to 2% w/w total degradation products after storage at room temperature for at least 2 years, or 0.000001% to 1.5% w/w total degradation products after storage at room temperature for at least 2 years, or 0.000001% to 1% w/w total degradation products after storage at room temperature for at least 2 years, or 0.000001% to 0.5% total degradation products after storage at room temperature for at least 2 years.

In some embodiments, an oral dosage form of the disclosure comprising about 5% w/w low peroxide grade povidone exhibits at least about 40% decrease in total degradation products as compared to an oral dosage form that comprises povidone that is not low peroxide grade povidone. In some embodiments, an oral dosage form of the disclosure comprising about 5% w/w low peroxide grade povidone exhibits at least about 50% decrease in total degradation products as compared to an oral dosage form that comprises povidone that is not low peroxide grade povidone. In some embodiments, an oral dosage form of the disclosure comprising about 5% w/w low peroxide grade povidone exhibits at least about 60% decrease in total degradation products as compared to an oral dosage form that comprises povidone that is not low peroxide grade povidone. In some embodiments, an oral dosage form of the disclosure comprising about 5% w/w low peroxide grade povidone exhibits at least about 70% decrease in total degradation products as compared to an oral dosage form that comprises povidone that is not low peroxide grade povidone. In some embodiments, an oral dosage form of the disclosure comprising about 5% w/w low peroxide grade povidone exhibits at least about 80% decrease in total degradation products as compared to an oral dosage form that comprises povidone that is not low peroxide grade povidone. In some embodiments, the low grade povidone contains between 0 ppm and 50 ppm peroxides, or between 0 ppm and 25 ppm peroxides.

In some embodiments, methods of making oral dose forms are provided that include the step of combining the deuruxolitinib phosphate with intragranular microcrystalline cellulose, lactose monohydrate, hydroxypropyl cellulose, and polyvinylpyrrolidone. In such embodiments, the combining step may be followed by wet granulation to form particles, and then blending the particles with extragranular microcrystalline cellulose, colloidal silicon dioxide and magnesium stearate to form a blend. In such embodiments, the method includes a step of compressing the blend into a tablet.

In a non-limiting exemplary formulation, a tablet formulation is produced containing deuruxolitinib phosphate and the following inactive ingredients: microcrystalline cellulose, lactose monohydrate, polyvinylpyrrolidone, hydroxypropyl cellulose, colloidal silicon dioxide, and magnesium stearate. For instance, for each tablet, 10.5 mg of deuruxolitinib phosphate is combined with 39.9 mg microcrystalline cellulose (intragranular), 36 mg lactose monohydrate, and 3.6 mg hydroxypropyl cellulose and mixed in a higher shear granulator. Then, 6.0 mg aqueous polyvinylpyrrolidone (e.g., povidone K30 or povidone K30 LP) is added during mixing to form particles. The particles are dried at about 60° C. and then milled. The resulting milled granules are mixed with 22.8 mg microcrystalline cellulose (extragranular), 0.6 mg colloidal silicon dioxide, and 0.6 mg magnesium stearate in a mixer to form the final blend. The final blend is compressed into tablets using a rotary press. The tablets may then be coated with a high performance moisture barrier film coating using art-standard coating techniques.

In some embodiments, a tablet comprises the following ingredients:

TABLE 1a
8 mg strength deuruxolitinib tablet (contains
equivalent of 8 mg free base deuruxolitinib).

| Component | Wt % | Amount (mg) |
|---|---|---|
| Deuruxolitinib phosphate | 8.75 | 10.5 |
| Microcrystalline cellulose | 33.25 | 39.90 |
| (intragranular) | | |
| Microcrystalline cellulose | 19 | 22.80 |
| (extragranular) | | |
| Lactose monohydrate | 30 | 36.00 |
| Polyvinylpyrrolidone (povidone) | 5 | 6.00 |
| Hydroxypropyl cellulose | 3 | 3.60 |
| Colloidal silicon dioxide | 0.5 | 0.60 |
| Magnesium stearate | 0.5 | 0.60 |
| Purified water | As required | As required |
| Total (tablet core) | 100 | 120 |
| Opadry ® amb II | | 3.60 |
| Total (coated tablet) | | 123.6 |

In some embodiments, a tablet comprises the following ingredients:

TABLE 1b
8 mg strength deuruxolitinib tablet (contains
equivalent of 8 mg free base deuruxolitinib).

| Component | Wt % | Amount (mg) |
|---|---|---|
| Deuruxolitinib phosphate | 8.75 | 10.5 |
| Microcrystalline cellulose (intragranular) | 33.25 | 39.90 |
| Microcrystalline cellulose (extragranular) | 19 | 22.80 |
| Lactose monohydrate | 30 | 36.00 |
| Polyvinylpyrrolidone (Kollidon ® 30 LP) | 5 | 6.00 |
| Hydroxypropyl cellulose | 3 | 3.60 |
| Colloidal silicon dioxide | 0.5 | 0.60 |
| Magnesium stearate | 0.5 | 0.60 |
| Purified water | As required | As required |
| Total (tablet core) | 100 | 120 |
| Opadry ® amb II | | 3.60 |
| Total (coated tablet) | | 123.6 |

In such embodiments, the total weight of the tablet core is about 120 mg and the dose of deuruxolitinib phosphate is the equivalent of 8 mg free base. In some embodiments, the tablet is coated with, for example, 3.60 mg Opadry® amb II coating. In some embodiments, the tablet film coating contains excipients such as: carmine, FD&C blue #2 aluminum lake, glyceryl mono and dicaprylocaprate, polyvinyl alcohol, sodium lauryl sulfate, talc, and titanium dioxide.

In some embodiments, the oral dosage form of the disclosure (e.g., a tablet) contains about 1 mg to about 500 mg deuruxolitinib phosphate, or about 1 mg to about 450 mg deuruxolitinib phosphate, or about 1 mg to about 400 mg deuruxolitinib phosphate, or about 1 mg to about 350 mg deuruxolitinib phosphate, or about 1 mg to about 300 mg deuruxolitinib phosphate, or about 1 mg to about 250 mg deuruxolitinib phosphate, or about 1 mg to about 200 mg deuruxolitinib phosphate, or about 1 mg to about 150 mg deuruxolitinib phosphate, or about 1 mg to about 100 mg deuruxolitinib phosphate, or about 1 mg to about 90 mg deuruxolitinib phosphate, or about 1 mg to about 85 mg deuruxolitinib phosphate, or about 1 mg to about 80 mg deuruxolitinib phosphate, or about 1 mg to about 75 mg deuruxolitinib phosphate, or about 1 mg to about 70 mg deuruxolitinib phosphate, or about 1 mg to about 65 mg deuruxolitinib phosphate, or about 1 mg to about 60 mg deuruxolitinib phosphate, or about 1 mg to about 55 mg deuruxolitinib phosphate, or about 1 mg to about 50 mg deuruxolitinib phosphate, or about 1 mg to about 45 mg deuruxolitinib phosphate, or about 1 mg to about 40 mg deuruxolitinib phosphate, or about 1 mg to about 35 mg deuruxolitinib phosphate, or about 1 mg to about 30 mg deuruxolitinib phosphate, or about 1 mg to about 25 mg deuruxolitinib phosphate, or about 1 mg to about 20 mg deuruxolitinib phosphate, or about 1 mg to about 15 mg deuruxolitinib phosphate, or about 1 mg to about 10 mg deuruxolitinib phosphate, or about 4 mg to about 200 mg deuruxolitinib phosphate, or about 4 mg to about 150 mg deuruxolitinib phosphate, or about 4 mg to about 100 mg deuruxolitinib phosphate, or about 4 mg to about 96 mg deuruxolitinib phosphate, or about 4 mg to about 92 mg deuruxolitinib phosphate, or about 4 mg to about 88 mg deuruxolitinib phosphate, or about 4 mg to about 84 mg deuruxolitinib phosphate, or about 4 mg to about 80 mg deuruxolitinib phosphate, or about 4 mg to about 76 mg deuruxolitinib phosphate, or about 4 mg to about 72 mg deuruxolitinib phosphate, or about 4 mg to about 68 mg deuruxolitinib phosphate, or about 4 mg to about 64 mg deuruxolitinib phosphate, or about 4 mg to about 60 mg deuruxolitinib phosphate, or about 4 mg to about 56 mg deuruxolitinib phosphate, or about 4 mg to about 52 mg deuruxolitinib phosphate, or about 4 mg to about 48 mg deuruxolitinib phosphate, or about 4 mg to about 36 mg deuruxolitinib phosphate, or about 4 mg to about 24 mg deuruxolitinib phosphate, or about 4 mg to about 16 mg deuruxolitinib phosphate, or about 4 mg to about 8 mg deuruxolitinib phosphate, or about 8 mg to about 100 mg deuruxolitinib phosphate, or about 8 mg to about 68 mg deuruxolitinib phosphate, or about 16 mg to about 100 mg deuruxolitinib phosphate, or about 16 mg to about 68 mg deuruxolitinib phosphate, or about 4 mg, or about 8 mg, or about 16 mg, or about 24 mg, or about 36 mg, or about 58 mg, or about 56 mg, or about 60 mg, or about 64 mg, or about 68 mg, or about 72 mg, or about 80 mg, or about 84 mg, or about 88 mg, or about 92 mg, or about 96 mg, or about 100 mg, or about 125 mg, or about 150 mg, or about 10 mg, or about 15 mg, or about 20 mg, or about 25 mg, or about 30 mg, or about 35 mg, or about 40 mg, or about 45 mg, or about 50 mg, or about 60 mg, or about 70 mg, or about 80 mg, or about 90 mg deuruxolitinib phosphate (fee base equivalent weight).

In some embodiments, the oral dosage forms of the disclosure are in the form of tablets exhibiting stability of at least 2 years at room temperature. In some embodiments, the assay of the deuruxolitinib is 75% to 125% of label claim, 85% to 115% of label claim, 90%-110% of label claim, or 95% to 105% of label claim after storage at room temperature for at least 2 years. In some embodiments, the assay of the deuruxolitinib is 85% to 115% of label claim, 90% to 110% of label claim, or 95% to 115% of label claim after storage at room temperature for at least 1 year. e.g., 1 year 1.1 years, 1.2 years, 1.3 years, 1.4 years, 1.5 years, 1.6 years, 1.7 years, 1.8 years, 1.9 years, 2 years.

In some embodiments, the oral dosage forms of the disclosure are administered to a subject suffering from a disease, condition, or disorder that can be treated by compounds that modulate the activity of Janus Associated Kinase 1 (JAK1) and/or Janus Associated Kinase 2 (JAK2). Such diseases, conditions or disorders include, without limitation, skin diseases such as proliferative, autoimmune and/or inflammatory skin disorders including psoriasis, atopic dermatitis, scleroderma, rosacea, skin cancers, dermatitis, dermatitis herpetiformis, dermatomyositis, vitiligo, hair loss disorders, contact dermatitis, xerosis, ichthyosis, hidradenitis suppurativa, urticaria, lichen planus, prurigo nodularis, vasculitis, cutaneous lupus erythematosus (CLE), and chronic idiopathic pruritus; hyperproliferative disorders or cancers including polycythemia vera, essential thrombocytopenia, and myelofibrosis; respiratory diseases such as asthma, chronic obstructive pulmonary disease, chronic lung allograft dysfunction, e.g., bronchiolitis obliterans syndrome, pulmonary fibrosis, cystic fibrosis, rhinitis, bronchiolitis, byssinosis, pneumoconiosis, bronchiectasis, hypersensitivity pneumonitis, lung cancers, mesothelioma and sarcoidosis; gastrointestinal diseases such as inflammatory bowel disease, ulcerative colitis, Crohn's disease, retroperitoneal fibrosis, celiac disease and cancers; eye diseases such as myasthenia gravis, Sjogren's syndrome, conjunctivitis, scleritis, uveitis, dry eye syndrome, keratitis, iritis; systemic indications such as lupus, multiple sclerosis, rheumatoid arthritis, type I diabetes and complications from diabetes, cancers, ankylosing spondylitis and psoriatic arthritis; as well as other autoimmune diseases and indications where immunosuppression would be desirable, for example, to treat or prevent acute and/or chronic graft-versus-host disease (e.g., in organ transplantation). In certain embodiments, a disease or condition mediated alone, or in part, by Janus Associated Kinases (JAKs) is a hair loss disorder, such as alopecia areata.

In some embodiments, a method of treating or preventing a hair loss disorder in a subject (such as alopecia areata) is provided and includes administering to the subject an oral dosage form containing deuruxolitinib phosphate at a dose of 4 mg to 48 mg (free base equivalent weight). In certain embodiments, the deuruxolitinib phosphate is administered to the subject at a dose of about 10.5 mg, twice per day, which is the equivalent of 8 mg, twice per day, or 16 mg/day of the free base form of deuruxolitinib. In some embodiments, the subject is a mammalian subject. In some embodiments, the subject is a human subject. In some embodiments, the oral dosage form of deuruxolitinib phosphate is a tablet containing about 10.5 mg of deuruxolitinib phosphate.

In some embodiments, a method of treating or preventing a disease associated with JAK1 or JAK2 is provided and includes administering to the subject an oral dosage form containing deuruxolitinib phosphate at a dose of 1 mg to 500 mg (free base equivalent weight). In some embodiments, the deuruxolitinib phosphate is administered to the subject at a dose of 1 mg to 250 mg, 1 mg to 150 mg, 1 mg to 100 mg, 1 mg to 50 mg, 5 mg to 150 mg, 5 mg to 100 mg, 5 mg to 50 mg, 10 mg, 20 mg, 30 mg, 40 mg, 50 mg, 60 mg, 70 mg, 80 mg, 90 mg, 100 mg, 4 mg, 8 mg, 16 mg, 24 mg, 36 mg, 48 mg, 52 mg, 56 mg, 60 mg, 64 mg, 68 mg, 72 mg, 76 mg, 80 mg, 84 mg, 88 mg, 92 mg, or 96 mg (free base equivalent weight). In some embodiments, the deuruxolitinib phosphate is administered to the subject at a dose of about 4 mg/day to about 100 mg/day, or about 4 mg/day to about 50 mg/day, or about 8 mg/day to about 48 mg/day, or about 16 mg/day to about 48 mg/day.

Various modifications of the disclosure in addition to those shown and described herein will become apparent to those skilled in the art from the foregoing description and fall within the scope of the appended claims. Preferred features of each aspect of the disclosure are as for each of the other aspects mutatis mutandis. The documents referred to herein are incorporated by reference to the fullest extent permitted by law.

### EXAMPLES

### Example 1

### Preparation of 8 mg Strength Deuruxolitinib Tablets (Contain Equivalent of 8 mg Free Base Deuruxolitinib)

TABLE 1
8 mg strength deuruxolitinib tablet composition

| Component | Wt % |
|---|---|
| Deuruxolitinib phosphate | 8.75 |
| Microcrystalline cellulose | 33.25 |
| (intragranular) | |
| Microcrystalline cellulose | 19 |
| (extragranular) | |
| Lactose monohydrate | 30 |
| Polyvinylpyrrolidone (povidone) | 5 |
| Hydroxypropyl cellulose | 3 |
| Colloidal silicon dioxide | 0.5 |
| Magnesium stearate | 0.5 |
| Purified water | As required^{a} |
| Total (tablet core) | 100 |
| Opadry ® amb II | |
| Purified water | As required |
| Total (coated tablet) | |

^{a}Removed during processing

### Step 1: High-Shear Wet Granulation

Povidone K30 or povidone K30 LP was dissolved in purified water to prepare a 15% w/w povidone in water binder solution. For instance, in some embodiments, povidone K30 LP was dissolved in purified water to prepare 15% w/w povidone in water binder solution.

Lactose monohydrate, deuruxolitinib phosphate, low-substituted hydroxypropyl cellulose, and microcrystalline cellulose were screened and added to a 600-L high-shear granulator bowl. The materials were dry mixed for 5 minutes with the impeller at approximately 100 rpm. After completion of dry mixing, addition of the binder solution was started with the impeller at approximately 100 rpm and chopper at approximately 1500 rpm. Granulation continued until all of the binder solution was added, approximately 8 minutes. The material was wet massed until end-point consistency is reached, approximately 1 minute.

### Step 2: Fluid Bed Drying

A fluid bed dryer (670-L capacity) was pre-warmed to approximately 70° C. The wet granules were transferred to the warmed bowl and dried until the target loss on drying (LOD) has been reached (LOD≤3.0% w/w).

### Step 3: Milling

The dried granules were passed through a conical mill fitted with a 0.040″ grated screen.

### Step 4: Blending

The dried, milled granules were loaded into a 10 cu. ft. V-shell. Colloidal silicon dioxide and microcrystalline cellulose were screened and added to the V-shell. The materials were blended in the V-blender for 200 rotations.

A portion of the blended material was removed from the V-shell. Magnesium stearate was passed through a screen to de-lump and mixed with the removed portion of blended material. This combined portion was then charged back to the V-shell. The materials were blended in the V-blender for 25 rotations.

### Step 5: Compression

The final blend was transferred to the hopper on a rotary tablet press with a forced feeder. Tablets were compressed using 6.5 mm round tooling to an average weight of approximately 120 mg and average hardness of 4.5-7.5 kp. Tablets were passed through a de-duster and a metal detector.

### Step 6: Coating

Opadry® amb II Purple was dispersed in purified water to prepare a 20% w/w solids coating suspension.

A tablet coater with 48″ perforated pan was pre-warmed to approximately 50° C. The tablet cores were transferred to the warmed pan and jogged until the exhaust temperature reaches 49±1° C. The coating suspension was sprayed until the target % weight gain of 3.0±0.5 was achieved. The coated tablets were dried in the coating pan for 5 minutes under the same conditions and then cooled in the coating pan until the exhaust temperature is ≤33° C.

### Example 2

### Stability Testing on 8 mg Strength Deuruxolitinib Phosphate Tablets (Contain Equivalent 8 Mg Free Base Deuruxolitinib).

The stability of 8 mg strength, coated deuruxolitinib phosphate tablets (formulated according to Table 1) was tested in aqueous media for 48 hours at 37° C. Briefly, samples were prepared in triplicate for stability testing in buffer solutions of pH 1.0, 3.2, 4.2, 5.2, 6.8, simulated gastric fluid (SGF), and simulated intestinal fluid (SIF) using the shake-flask method. Assay and degradation products were measured by HPLC after 24 and 48 hours of storage to evaluate solution stability. The stability results are shown in Table 2 below. After 48 hours, no greater than 0.5% total degradation products were detected across all pH media tested indicating good stability of the oral dosage form.

TABLE 2
Stability results for deuruxolitinib
phosphate tablets (48 hours at 37° C.)

| Buffer Solution | Mean Deuruxolitinib Total Degradation Products |
|---|---|
| (Target pH) | (% Area) |
| 1.0 | None detected |
| 3.2 | None detected |
| 4.2 | 0.1 |
| 5.2 | 0.5 |
| 6.8 | 0.2 |
| SGF | None detected |
| SIF | 0.1 |

The impact of binder excipients on the stability of 8 mg deuruxolitinib phosphate tablets was tested using different grades of polyvinylpyrrolidone (povidone) as the binder and storing the resulting tablets at 40 BC/75 RH (open container) or 60° C. (closed container) for 4 weeks. For the 8 mg strength deuruxolitinib phosphate dosage forms, 5% povidone K30, 5% povidone K-29/32, or 5% povidone K30 LP (low peroxide) grades were used as the binder. Degradation products were measured by HPLC. A deuruxolitinib phosphate reference standard along with reference standards for impurities were used to determine levels of impurities (i.e., “degradation products”) based on relative retention time. As shown in Table 3, less than 0.1% total degradation products were detected in tablets containing the low peroxide grade povidone even at elevated temperatures and humidity.

TABLE 3
Excipient compatibility results - 4-week study
at elevated temperature and/or humidity

Total Degradation Products
(% Area)

| | | 60° C., | 40° C./75% |
|---|---|---|---|
| Excipient | Function | Closed | RH, Open |
| Povidone K30 | Binder | ND | 0.5 |
| Povidone K30 LP | Binder | ND | <0.1 |
| Povidone K-29/32 | Binder | ND | 1.4 |

ND = None detected (<0.05% area)

The storage stability of the 8 mg strength deuruxolitinib phosphate tablets (containing povidone K30) was examined after storage for up to 10 months in unsealed bottles at 40° C./75% RH. Four degradation products were detected using HPLC that eluted at 0.37, 0.56, 0.77, and 0.89 relative to deuruxolitinib phosphate. The detection threshold was 0.05% w/w. As shown in Table 4, two of these degradation products RRT 0.56 and CTP-543-ZH were detectable at 0.08% w/w at 10 months, but below the threshold prior to that time.

TABLE 4
Individual degradation products from deuruxolitinib
phosphate tablets, 8 mg, at 40 C./75% RH (Open)

| Degradation | Individual Degradation Product Levels (% w/w) at Intervals (Months) | | | | |
|---|---|---|---|---|---|
| Product | 0 | 1 | 2 | 3 | 10 |
| RRT 0.37 | NR | NR | NR | 0.06 | 0.15 |
| RRT 0.56 | NR | NR | NR | NR | 0.08 |
| RRT 0.77 | 0.19 | 0.25 | 0.28 | 0.31 | 0.44 |
| (CTP-543-ZB) | | | | | |
| RRT 0.89 | NR | NR | NR | NR | 0.08 |
| (CTP-543-ZH) | | | | | |

NR = None reportable; reporting threshold is 0.05%

The storage stability of the 8 mg strength deuruxolitinib tablets (containing povidone K30 LP) was examined after storage for up to 18 months in 60 cc HDPE bottles at 25° C./60% RH, up to 12 months in 60 cc HDPE bottles at 30° C./65% RH, or up to 6 months in 60 cc HDPE bottles at 40° C./75% RH. Representative results from one of the test batches is summarized in Table 5-7. The results indicated that low peroxide grade povidone resulted in less overall degradation products as compared to povidone (normal peroxide content). Further, at least one of the degradation products, CTP-543-ZH, remained below the detectable threshold, even after a year of storage. Therefore, deuruxolitinib phosphate coated tablets containing low peroxide povidone had significantly less degradation products as compared to deuruxolitinib phosphate coated tablets containing povidone with higher hydrogen peroxide levels.

TABLE 5
Stability data for deuruxolitinib phosphate tablets, 8 mg, at 25° C./60% RH

| | Acceptance | Time Point (months) | | | | | | |
|---|---|---|---|---|---|---|---|---|
| Test Name | Criteria | 0 | 1 | 3 | 6 | 9 | 12 | 18 |
| Individual | CTP-543-ZB: ≤0.50 | 0.08 | 0.07 | 0.07 | 0.07 | 0.08 | 0.08 | 0.07 |
| Degradation | CTP-543-ZH: ≤0.50 | NR | NR | NR | NR | NR | NR | NR |
| Products (% | Unspecified: ≤0.20 | NR | NR | NR | NR | NR | NR | NR |
| w/w) | | | | | | | | |
| Total | ≤2.0 | 0.1 | 0.1 | 0.1 | 0.1 | 0.1 | 0.1 | 0.1 |
| Degradation | | | | | | | | |
| Products | | | | | | | | |
| (% w/w) | | | | | | | | |
| Assay (% | 90-110 | 103 | 102 | 101 | 102 | 101 | 102 | 103 |
| Label | | | | | | | | |
| Claim) | | | | | | | | |
| Enantiomeric | CTP-543-X: ≤0.90 | 0.18 | 0.18 | 0.17 | 0.17 | 0.16 | 0.17 | 0.18 |
| Purity | | | | | | | | |
| (% Area) | | | | | | | | |
| Tablet | Report result | 7.0 | 6.9 | 6.9 | 7.1 | 6.8 | 6.8 | 6.3 |
| Breaking | | | | | | | | |
| Force (kp) | | | | | | | | |
| Water | Report result | 4.2 | 3.6 | 3.2 | 4.3 | 4.3 | 3.7 | 3.4 |
| Content (% | | | | | | | | |
| w/w) | | | | | | | | |
| Total | Maximum | <20 | NT | NT | NT | NT | <20 | NT |
| Aerobic | Acceptable Count = | | | | | | | |
| Microbial | 2000 | | | | | | | |
| Count | | | | | | | | |
| (CFU/g) | | | | | | | | |
| Total | Maximum | <20 | NT | NT | NT | NT | <20 | NT |
| Combined | Acceptable Count = | | | | | | | |
| Yeasts and | 200 | | | | | | | |
| Molds | | | | | | | | |
| Count | | | | | | | | |
| (CFU/g) | | | | | | | | |
| Specified | Absent | Absent | NT | NT | NT | NT | Absent | NT |
| Microorganisms: | | | | | | | | |
| E. coli | | | | | | | | |

NR = None reportable; reporting threshold is 0.05%
NT = Not a scheduled test, as per protocol

TABLE 6
Stability data for deuruxolitinib phosphate tablets, 8 mg, at 30° C./65% RH

| | | Time Point (months) | | | | |
|---|---|---|---|---|---|---|
| Test Name | Acceptance Criteria | 0 | 3 | 6 | 9 | 12 |
| Individual | CTP-543-ZB: ≤0.50 | 0.08 | 0.07 | 0.08 | 0.08 | 0.08 |
| Degradation | CTP-543-ZH: ≤0.50 | NR | NR | NR | NR | NR |
| Products (% | Unspecified: ≤0.20 | NR | NR | NR | NR | NR |
| w/w) | | | | | | |
| Total | ≤2.0 | 0.1 | 0.1 | 0.1 | 0.1 | 0.1 |
| Degradation | | | | | | |
| Products | | | | | | |
| (% w/w) | | | | | | |
| Assay (% Label | 90-110 | 103 | 101 | 101 | 102 | 102 |
| Claim) | | | | | | |
| Enantiomeric | CTP-543-X: ≤0.90 | 0.18 | 0.17 | 0.17 | 0.17 | 0.17 |
| Purity (% Area) | | | | | | |
| Tablet Breaking | Report result | 7.0 | 7.0 | 6.6 | 6.4 | 6.6 |
| Force (kp) | | | | | | |
| Water Content | Report result | 4.2 | 3.3 | 4.3 | 4.1 | 4.1 |
| (% w/w) | | | | | | |
| Total Aerobic | Maximum Acceptable | <20 | NT | NT | NT | <20 |
| Microbial Count | Count = 2000 | | | | | |
| (CFU/g) | | | | | | |
| Total Combined | Maximum Acceptable | <20 | NT | NT | NT | <20 |
| Yeasts and | Count = 200 | | | | | |
| Molds Count | | | | | | |
| (CFU/g) | | | | | | |
| Specified | Absent | Absent | NT | NT | NT | Absent |
| Microorganisms: | | | | | | |
| E. coli | | | | | | |

NR = None reportable; reporting threshold is 0.05%
NT = Not a scheduled test, as per protocol

TABLE 7
Stability data for deuruxolitinib phosphate tablets, 8 mg, at 40° C./75% RH

| | | Time Point (months) | | | | |
|---|---|---|---|---|---|---|
| Test Name | Acceptance Criteria | 0 | 1 | 2 | 3 | 6 |
| Individual | CTP-543-ZB: ≤0.50 | 0.08 | 0.08 | 0.08 | 0.08 | 0.09 |
| Degradation Products | CTP-543-ZH: ≤0.50 | NR^{a} | NR | NR | NR | NR |
| (% w/w) | Unspecified: ≤0.20 | NR^{a} | NR | NR | NR | NR |
| Total Degradation | ≤2.0 | 0.1 | 0.1 | 0.1 | 0.1 | 0.1 |
| Products | | | | | | |
| (% w/w) | | | | | | |
| Assay (% Label | 90-110 | 103 | 101 | 101 | 102 | 101 |
| Claim) | | | | | | |
| Enantiomeric Purity | CTP-543-X: ≤0.90 | 0.18 | 0.17 | 0.18 | 0.17 | 0.17 |
| (% Area) | | | | | | |
| Tablet Breaking Force | Report result | 7.0 | 7.0 | 6.8 | 6.7 | 6.0 |
| (kp) | | | | | | |
| Water Content (% | Report result | 4.2 | 3.5 | 3.1 | 3.3 | 4.3 |
| w/w) | | | | | | |
| Total Aerobic | Maximum Acceptable | <20 | NT | NT | NT | <20 |
| Microbial Count | Count = 2000 | | | | | |
| (CFU/g) | | | | | | |
| Total Combined | Maximum Acceptable | <20 | NT | NT | NT | <20 |
| Yeasts and Molds | Count = 200 | | | | | |
| Count (CFU/g) | | | | | | |
| Specified | Absent | Absent | NT | NT | NT | Absent |
| Microorganisms: | | | | | | |
| E. coli | | | | | | |

NR = None reportable; reporting threshold is 0.05%
NT = Not a scheduled test, as per protocol

The present disclosure has been described with respect to representative examples that are to be considered illustrative embodiments and do not limit the scope of the disclosure which is defined solely by the claims. All references to publications, including scientific publications, treatises, textbooks, patent applications, and issued patents are hereby incorporated by reference for all purposes.

We claim:

1. An oral dosage form comprising deuruxolitinib phosphate and pharmaceutically acceptable carrier material, wherein the dosage form comprises less than 0.05% wt/wt of CTP-543-ZH.

2. The oral dosage form of claim 1, wherein the pharmaceutically acceptable carrier material comprises microcrystalline cellulose, lactose monohydrate, polyvinylpyrrolidone, hydroxypropyl cellulose, colloidal silicon dioxide and magnesium stearate.

3. An oral dosage form of claim 2, comprising:
a. about 8.75% w/w deuruxolitinib phosphate;
b. about 50% to about 60% w/w microcrystalline cellulose;
c. about 25% to about 35% w/w lactose monohydrate;
d. about 3% to about 6% w/w polyvinylpyrrolidone;
e. about 2% to about 4% w/w hydroxypropyl cellulose;
f. about 0.25% to about 0.75% w/w colloidal silicon dioxide; and
g. about 0.25% to about 0.75% w/w magnesium stearate.

4. An oral dosage form of claim 2, comprising:
a. about 8.75% w/w deuruxolitinib phosphate;
b. about 52% w/w microcrystalline cellulose;
c. about 30% w/w lactose monohydrate;
d. about 5% w/w polyvinylpyrrolidone;
e. about 3% w/w hydroxypropyl cellulose;
f. about 0.5% w/w colloidal silicon dioxide; and
g. about 0.5% w/w magnesium stearate.

5. The oral dosage form of claim 1, wherein the pharmaceutically acceptable carrier material comprises a low peroxide grade polyvinylpyrrolidone.

6. The oral dosage form of claim 2, wherein the microcrystalline cellulose is in intragranular form and extragranular form.

7. The oral dosage form of claim 6, wherein the oral dosage form comprises about 30% w/w to about 40% w/w intragranular microcrystalline and about 18% w/w to about 25% w/w extragranular microcrystalline cellulose.

8. The oral dosage form of claim 7, wherein the oral dosage form comprises about 33%-34% w/w intragranular microcrystalline cellulose and about 19% w/w extragranular microcrystalline cellulose.

9. The oral dosage form of claim 1, wherein the oral dosage form does not comprise sodium starch glycolate.

10. The oral dosage form of claim 2, wherein the polyvinylpyrrolidone is a low peroxide grade polyvinylpyrrolidone.

11. The oral dosage form of claim 1, wherein the oral dosage form is in a tablet.

12. The oral dosage form of claim 1, wherein the oral dosage form is suitable for twice a day administration.

13. The oral dosage form of claim 1, wherein the dosage form comprises less than 0.05% wt/wt CTP-543-ZH after storage for 1 week at 25° C. at 50% relative humidity.

14. The oral dosage form of claim 1, wherein the dosage form comprises less than 0.05% wt/wt CTP-543-ZH after storage for 1 week at 40° C. at 75% relative humidity.

15. The oral dosage form of claim 1, wherein the dosage form comprises less than 0.05% wt/wt CTP-543-ZH after storage for 6 months at 25° C. at 50% relative humidity.

16. The oral dosage form of claim 1, wherein the dosage form comprises less than 0.05% wt/wt CTP-543-ZH after storage for 6 months at 40° C. at 75% relative humidity.

17. The oral dosage form of claim 1, wherein the dosage form comprises less than 0.01% wt/wt CTP-543-ZH.

18. The oral dosage form of claim 17, wherein the dosage form comprises less than 0.01% wt/wt CTP-543-ZH after storage for 1 week at 25° C. at 50% relative humidity.

19. The oral dosage form of claim 17, wherein the dosage form comprises less than 0.01% wt/wt CTP-543-ZH after storage for 1 week at 40° C. at 75% relative humidity.

20. The oral dosage form of claim 19, wherein the dosage form comprises less than 0.01% wt/wt CTP-543-ZH after storage for 6 months at 25° C. at 50% relative humidity.

21. The oral dosage form of claim 19, wherein the dosage form comprises less than 0.01% wt/wt CTP-543-ZH after storage for 6 months at 40° C. at 75% relative humidity.

22. A method of making the oral dosage form of claim 1, the method comprising
(a) combining deuruxolitinib phosphate, microcrystalline cellulose, lactose monohydrate, hydroxypropyl cellulose, and polyvinylpyrrolidone;
(b) wet granulating the combination of (a) to form particles;
(c) blending the particles formed with microcrystalline cellulose, colloidal silicon dioxide and magnesium stearate to form a blend; and
(d) compressing the blend into a tablet.

23. The method of claim 22, wherein the polyvinylpyrrolidone is a low peroxide grade polyvinylpyrrolidone.

24. A coated tablet comprising:
a. a tablet core, wherein the tablet core comprises
i. about 8.75% w/w deuruxolitinib phosphate;
ii. about 52% w/w microcrystalline cellulose;
iii. about 30% w/w lactose monohydrate;
iv. about 5% w/w polyvinylpyrrolidone;
v. about 3% w/w hydroxypropyl cellulose;
vi. about 0.5% colloidal silicon dioxide; and
vii. about 0.5% magnesium stearate; and
b. an outer coating layer.

25. The coated tablet of claim 24, wherein the polyvinylpyrrolidone is a low peroxide grade polyvinylpyrrolidone.

26. The coated tablet of claim 24, wherein the outer coating layer comprises Opadry® amb II.

27. A method of treating a hair loss disorder in a human subject, the method comprising administering to the human subject the oral dosage form of claim 1.

28. The method of claim 27, wherein the hair loss disorder is alopecia areata.

29. The method of claim 28, wherein the oral dosage form is administered twice a day.
